(19) United States
(12) Patent Application Publication
(10) Pub. No.: US 2023/0212602 A1
(43) Pub. Date: Jul. 6, 2023

(54) Use of insecticidal protein

(71) Applicant: BEIJING DABEINONG BIOTECHNOLOGY CO., LTD., Beijing (CN)

(72) Inventors: Tengyu CHANG, Beijing (CN); Qinyang WANG, Beijing (CN); Aihong ZHANG, Beijing (CN); Qing TAO, Beijing (CN)

(21) Appl. No.: 18/063,081

(22) Filed: Dec. 8, 2022

(30) Foreign Application Priority Data
Dec. 13, 2021 (CN) 202111515707.2

(51) Int. Cl. C12N 15/82

(52) U.S. Cl. CPC C12N 15/8286

(57) ABSTRACT

Related is a use of an insecticidal protein. The insecticidal protein may be used to control Monolepta hieroglyphica (Motschulsky). A method for controlling the Monolepta hieroglyphica (Motschulsky) includes: allowing the Monolepta hieroglyphica (Motschulsky) to be at least in contact with an ACh1 protein. In the present application, the Monolepta hieroglyphica (Motschulsky) is controlled through producing the ACh1 protein that can kill the Monolepta hieroglyphica (Motschulsky) in bacteria and/or a plant in vivo.

### CROSS-REFERENCE TO RELATED APPLICATION

The present application is based on and claims priority to Chinese application with a CN application number of 202111515707.2 and an application date of Dec. 13, 2021, the disclosure of which is hereby incorporated by reference again in its entirety.

### SEQUENCE LISTING

The instant application contains a Sequence Listing which has submitted electronically in ASCII format and is hereby incorporated by reference in its entirety. Said ASCII copy is named PN 192848 SEQ LIST.xml and is 32,647 bytes in size. The sequence listing contains 20 sequences, which is identical in substance to the sequences disclosed in the CN application and includes no new matter.

### TECHNICAL FIELD

The present disclosure relates to a use of an insecticidal protein, and in particular, to a use of an ACh1 protein for controlling damage of Monolepta hieroglyphica (Motschulsky) to a plant by expressing in the plant.

### BACKGROUND

Monolepta hieroglyphica (Motschulsky) is also known as monolepta and belongs to Coleoptera Chrysomelidae, which is a new type of pests that damage corns, and a holometabolous insect belonging to the galeruca of Coleoptera Chrysomelidae. Infestation mainly occurs in July to September. Corn leaves are damaged by adults. The leaves are mesh-like in less severe cases, and are entirely dried-up in severe cases. The pest has the habit of swarming and tends to feed on tender leaves, which usually eats the corn leaves from top to bottom by gathering on a plant. Tender leaves are bitten into holes, reticulate veins or epidermis is left after the middle and lower leaves are damaged, causing small irregular white spots seen from a distance, and having large impact on photosynthesis. After the tasseling and silking of corns, the pest likes to feed on anthers and filaments, which severely affects the normal flowering and pollination of the corns, and is easy to cause ear rot.

Corn and soybean are important food crops in China. The Monolepta hieroglyphica (Motschulsky) causes huge food losses each year, and even affects the living conditions of the local population. In order to control the Monolepta hieroglyphica (Motschulsky), main control methods usually used by people are agricultural control, chemical control, physical control and biological control.

The agricultural control is to comprehensively coordinate and manage an entire farmland ecosystem from multi-factors, and regulate crops, pests, and environmental factors so as to create a farmland ecological environment that facilitates crop growth but not facilitates the occurrence of the Monolepta hieroglyphica (Motschulsky). For example, broad irrigation is used to drown eggs and larvae in soil. However, this method has a little effect, and consumes a lot of water resources.

The chemical control is pesticide control, which uses chemical pesticides to kill pests and is an important part of the comprehensive management of the Monolepta hieroglyphica (Motschulsky). The chemical control has the characteristics of rapidity, convenience, simplicity and high economic benefits, and is an essential emergency measure, especially in the case of a large occurrence of the chemical control. At present, a chemical control method mainly includes using drug liquid spray and seed coating agents. The drug liquid spray starts to control when there are more than 50 pests in every 100 corn plants and spray a 2000 times solution of perchloride imidacloprid in a high-incidence season of the Monolepta hieroglyphica (Motschulsky). Waste cotton may also be used to dip 200-time phorate emulsion and is placed every 50 square meters to 100 square meters for fumigation. The dipped waste cotton is placed on the upper leaf sheath of the ear of a corn for control. However, the larvae of the Monolepta hieroglyphica (Motschulsky) live underground, so that chemical spraying cannot control the underground larvae. The adults of the Monolepta hieroglyphica (Motschulsky) can fly and jump, and migrate between fields and hosts, so that large-scale unified control and prevention is required. This requires a lot of organizational management costs and is extremely difficult to implement. Currently, small farmers only manage pesticide application of their own fields, so that the adults of the Monolepta hieroglyphica (Motschulsky) cannot be effectively controlled. When the seed coating agent is used for control, dry or wet seeds are coated by a pesticide composition containing a binder, so that protective layers with certain functions and covering strength is formed outside the seeds. The process is called seed coating, and the composition coated outside the seeds is called the seed coating agent. Since the seed coating agent contains pesticide ingredients, the underground pests that damage the seeds can be controlled to a certain extent. However, the pest control effect of the seed coating agent cannot be stably maintained due to the influence of time and rain environments. Generally, a persistent period of the seed coating agent is only one month. That is to say, according to corn sowing in Northeast China, the seed coating agent is most effective in May and starts to attenuate in June. Eggs of the Monolepta hieroglyphica (Motschulsky) tend to hatch at the end of May, and the larvae are infested in June. The best effective period of the seed coating agent is staggered from the development period of the larvae of the Monolepta hieroglyphica (Motschulsky), so that the larvae of the Monolepta hieroglyphica (Motschulsky) cannot be well controlled. In addition, the chemical control also has its limitations. For example, the improper use may often lead to adverse consequences such as the phytotoxicity of crops, the drug resistance of pests, the killing of natural enemies, and the pollution of the environment, so the farmland ecosystem is destroyed and pesticide residues pose a threat to the safety of humans and animals.

Physical control is mainly based on the response of pests to various physical factors in environmental conditions, using various physical factors such as light, electricity, color, temperature and humidity, as well as mechanical devices for trapping, radiation sterility and other methods to control pests. However, the adults of the Monolepta hieroglyphica (Motschulsky) have no obvious chromotropism, so that the adults cannot be trapped by hanging yellow boards to achieve the purpose of killing. The adults of the Monolepta hieroglyphica (Motschulsky) are active during the day, especially at high-temperature noon in summer, which cannot be lured by using light (the intensity of the light cannot exceed that of daylight and is not attractive), and the Monolepta hieroglyphica (Motschulsky) is dormant in the grass near the ground and the lower part of the corn, which does not accept temptation. Therefore, no physical control measures have been found to have a certain effect on the damage of the Monolepta hieroglyphica (Motschulsky).

The biological control is the use of some beneficial organisms or biological metabolites to control the number of pest populations in order to achieve a purpose of reducing or eliminating the pests, such as the selection of pesticides with low toxicity to the natural enemies, and the application of the pesticides is adjusted according to differences in the occurrence periods of the pests and natural enemies in the field, the application of the pesticides is avoided while the natural enemies occur in large numbers to protect the natural enemies. Its characteristics are that it is safe for the humans and animals, low in the pollution of the environment, and may achieve a long-term control purpose of certain pests; however, the effect is often unstable, and the same investment is required regardless of the severity of the Monolepta hieroglyphica (Motschulsky). Since the Monolepta hieroglyphica (Motschulsky) spawns underground, the biological control is not suitable for egg parasitic beneficial insects such as trichogramma. The adults of the Monolepta hieroglyphica (Motschulsky) belong to Coleoptera that can fly and jump, so that lady beetles belonging to the same Coleoptera are unable to stress the adults of the Monolepta hieroglyphica (Motschulsky). Therefore, no biological control measures have been found to have a certain effect on the damage of the Monolepta hieroglyphica (Motschulsky).

In order to solve the limitations of the agricultural control, the chemical control, physical control and the biological control in practical applications, scientists found that some insect-resistant transgenic plants may be obtained by transferring insect-resistant genes encoding insecticidal proteins into plants so as to control plant pests.

Pest-resistant crops have been developed by genetically engineering crops to introduce a Bacillus thuringiensis (Bt) protein into crops. For example, Cry1Ab is used to develop corns resistant to corn borer. At present, these genetically modified crops are widely used in agriculture and provide farmers with an environmentally friendly alternative to traditional insect control methods. Although the genetically modified crops have been shown to be quite effective against lepidopteran pests (the corn borer, bollworm, and the like), no genetically modified crops have been found that can control the Monolepta hieroglyphica (Motschulsky). The main reason for this is that no Bt protein has been found to be virulent to the Monolepta hieroglyphica (Motschulsky).

ACh1 is a new class of insecticidal proteins, which is completely different from the traditional Bt protein. By analyzing a protein secondary structure, the protein is speculated to belong to a β- pore forming protein. The mechanism of action of such proteins is generally enzymatic cleavage activation, binding with receptors, formation of oligomers, and pore-forming on membrane surfaces. The enzymatic cleavage activation in insect gut, receptor binding on the insect gut and a physicochemical environment in the insect gut determine whether the transmembrane pore can form in cell membranes of the insect gut. After such type of protein is secreted by the bacteria, it needs to be digested in a target body to form an active protein. The enzyme cleavage process is mainly performed at an amino-terminal or carboxyl-terminal of the protein, to turn the protein into an active fragment. The active fragment binds to a receptor on an epithelial cell membrane of the insect gut to form oligomer, and inserts into an gut membrane, so that a transmembrane pore appears on the cell membrane, and the osmotic pressure change and pH balance and the like inside and outside the cell membrane are destroyed, and the digestion process of the insects is disrupted, finally resulting in death of the insects.

The ACh1 protein has been reported to have inhibitory activity against silkworm and potato beetles. However, there is no report on the control of plant damage by the Monolepta hieroglyphica (Motschulsky) by producing transgenic plants expressing the ACh1 protein so far.

### SUMMARY

The present disclosure is intended to provide a use of an insecticidal protein, and for the first time provide a method for controlling Monolepta hieroglyphica (Motschulsky) by producing a transgenic plant expressing an ACh1 protein, to effectively overcome technical defects in agricultural control, chemical control, physical control and biological control in the prior arts.

In order to achieve the above objective, the present disclosure provides a method for controlling Monolepta hieroglyphica (Motschulsky), including allowing the Monolepta hieroglyphica to be at least in contact with an ACh1 protein.

Preferably, the ACh1 protein is present in a host cell that produces at least the ACh1 protein, and the Monolepta hieroglyphica (Motschulsky) is in contact with at least the ACh1 protein by ingesting the host cell.

More preferably, the ACh1 protein is present in a bacterium or a transgenic plant that produces at least the ACh1 protein. The Monolepta hieroglyphica (Motschulsky) is in contact with at least the ACh1 protein by ingesting the bacterium or a tissue of the transgenic plant. After contacting, the growth of the Monolepta hieroglyphica (Motschulsky) is inhibited and/or death is caused, so as to achieve the control of the damage of the Monolepta hieroglyphica (Motschulsky) to plants.

The transgenic plant may be in any growth stages.

The tissue of the transgenic plant is a root, a leaf, a stem, a tassel, an ear, an anther, or a filament.

The control of the damage of the Monolepta hieroglyphica (Motschulsky) to the plants does not vary with the planting location and/or the planting time.

The plant is soybean, wheat, barley, corns, tobacco, rice, rape, cotton, or sunflowers.

A step before the contacting step is to plant a plant containing a polynucleotide encoding the ACh1 protein.

On the basis of the above technical solution, the ACh1 protein is an ACh1_1 protein, an ACh1_2 protein, an ACh1_3 protein, or an ACh1_4 protein.

Preferably, the ACh1 protein has an amino acid sequence shown in SEQ ID NO:1, SEQ ID NO:2, SEQ ID NO:3, or SEQ ID NO:4.

On the basis of the above technical solution, the plant further includes at least a second nucleotide different from the nucleotide encoding the ACh1 protein.

Further, the second nucleotide encodes a Cry-like insecticidal protein, a Vip-like insecticidal protein, a protease inhibitor, lectin, α-amylase, or a peroxidase.

Preferably, the second nucleotide encodes a Cry3Bb protein.

Further, the Cry3Bb protein has an amino acid sequence shown in SEQ ID NO:13. The second nucleotide has a nucleotide sequence shown in SEQ ID NO:14.

Optionally, the second nucleotide is a dsRNA that inhibits an important gene in a target insect pest.

In order to achieve the above objective, the present disclosure further provides a use of an ACh1 protein for controlling Monolepta hieroglyphica (Motschulsky).

In order to achieve the above objective, the present disclosure further provides a method for producing a plant for controlling Monolepta hieroglyphica (Motschulsky), including introducing a polynucleotide sequence encoding an ACh1 protein into a genome of the plant.

In order to achieve the above objective, the present disclosure further provides a method for producing a plant seed for controlling Monolepta hieroglyphica (Motschulsky), including hybridizing a first plant obtained by the method with a second plant, so as to produce a seed containing a polynucleotide sequence encoding an ACh1 protein.

In order to achieve the above objective, the present disclosure further provides a method for cultivating a plant for controlling Monolepta hieroglyphica (Motschulsky), including: at least one plant seed is planted, and the genome of the plant seed includes a polynucleotide sequence encoding an ACh1 protein; the plant seed is grown into a plant, and the plant is grown under conditions that the Monolepta hieroglyphica (Motschulsky) is artificially inoculated and/or the hazard of the Monolepta hieroglyphica (Motschulsky) naturally occurs, and a plant that has an attenuated plant damage and/or has an increased plant yield compared with other plants that do not have the polynucleotide sequences encoding the ACh1 protein is harvested.

The “contact” in the present disclosure means that insects and/or pests touch, stay and/or feed on a plant, a plant organ, a plant tissue or a plant cell, and the plant, plant organ, plant tissue or plant cell may be to express the insecticidal protein in vivo, or the plant, plant organ, plant tissue or plant cell has the insecticidal protein on the surface and/or has a microorganism that produces the insecticidal protein.

A term “control” and/or “prevention” in the present disclosure means that the Monolepta hieroglyphica (Motschulsky) is in contact with at least the ACh1 protein, and the growth of the Monolepta hieroglyphica (Motschulsky) is inhibited and/or death is caused after the contact. Further, the Monolepta hieroglyphica (Motschulsky) is in contact with at least the ACh1 protein by ingesting the plant tissue, and after the contact, all or part of the Monolepta hieroglyphica (Motschulsky) is inhibited in growth and/or death is caused. The inhibition refers to sub-lethal, namely it is not lethal but may cause a certain effect in growth, behavior, physiology, biochemistry and tissue and other aspects, such as slow growth and/or stop. At the same time, the plant should be morphologically normal, and may be cultivated by a conventional method for consumption and/or generation of products. In addition, the plant and/or plant seed containing the polynucleotide sequence encoding the ACh1 protein for controlling the Monolepta hieroglyphica (Motschulsky), under the condition that the Monolepta hieroglyphica (Motschulsky) is artificially inoculated and/or the Monolepta hieroglyphica (Motschulsky) naturally occurs, has the reduced plant damage compared with non-transgenic wild-type plants, and the specific manifestations include, but are not limited to, improved stem resistance, and/or increased grain weight, and/or increased yield, and the like. The “control” and/or “prevention” effect of the ACh1 protein on the Monolepta hieroglyphica (Motschulsky) may exist independently, and may not be weakened and/or disappeared due to the presence of other substances that may “control” and/or “prevent” the Monolepta hieroglyphica (Motschulsky). Specifically, if any tissue of the transgenic plant (containing the polynucleotide sequence encoding the ACh1 protein) simultaneously and/or asynchronously exist with and/or produce the ACh1 protein and/or another substance that may control the Monolepta hieroglyphica (Motschulsky), the existence of the another substance neither affects the “control” and/or “prevention” effect of the ACh1 protein on the Monolepta hieroglyphica (Motschulsky), nor may cause the “control” and/or “prevention” effect to be completely and/or partially implemented by the another substance, which is independent of the ACh1 protein. Usually, in the field, the ingestion process of the plant tissue by the Monolepta hieroglyphica (Motschulsky) is short and difficult to observe with naked eyes. Therefore, under the condition that the Monolepta hieroglyphica (Motschulsky) is artificially inoculated and/or the Monolepta hieroglyphica (Motschulsky) naturally occurs, for example, any tissues of the transgenic plant (containing the polynucleotide sequence encoding the ACh1 protein) have the dead Monolepta hieroglyphica (Motschulsky), and/or the Monolepta hieroglyphica (Motschulsky) on which the growth is inhibited, and/or have the reduced plant damage compared with the non-transgenic wild-type plants, the method and/or the use of the present disclosure is achieved. That is to say, the method and/or the use for controlling the Monolepta hieroglyphica (Motschulsky) is achieved by allowing the Monolepta hieroglyphica to be at least in contact with the ACh1 protein.

In the present disclosure, the expression of the ACh1 protein in a transgenic plant may be accompanied by the expression of one or more Cry-like insecticidal proteins and/or Vip-like insecticidal proteins. Co-expression of such more than one insecticidal toxin in the same transgenic plant may be achieved by genetically engineering the plant to contain and express a desired gene. In addition, one plant (first parent) may express the ACh1 protein by a genetic engineering operation, and a second plant (second parent) may express the Cry-like insecticidal proteins and/or Vip-like insecticidal proteins by the genetic engineering operation. Offspring plants expressing all the genes introduced into the first and second parents are obtained by hybridizing the first and second parents.

RNA interference (RNAi) refers to a phenomenon that is highly conserved during the evolution process and induced by a double-stranded RNA (dsRNA), and a homologous mRNA is efficiently and specifically degraded. Therefore, an RNAi technology may be used in the present disclosure to specifically knock out or shut down the expression of a specific gene in the target insect pest.

The Monolepta hieroglyphica (Motschulsky) in the present disclosure is a holometabolous insect belonging to the galeruca of Coleoptera Chrysomelidae. The adult body is 3.6-4.8 mm long, 2-2.5 mm wide, with 11 filamentous antennae of which ends are black and lengths are ⅔ of the body length; compound eyes are large and oval; the width of pronotum is greater than the length, and the surface is raised, and densely covered with many small punctation; a scutellum is black and triangular; the elytrum is distributed with linear fine punctation, half of each elytrum has 1 near-circular pale spot with black periphery, the posterolateral side of the pale spot is not completely closed, and the rear ends of the two wings are closed into a circle. The simple identification method is that there are large faint yellow spots on the two elytra of the adult, respectively, the periphery of the spot is black, and half of the elytrum end is yellow.

The Monolepta hieroglyphica (Motschulsky) is widely distributed in our country, mainly in Northeast China and North China, and provinces such as Jiangsu, Zhejiang, Hubei, Jiangxi, Fujian, Guangdong, Guangxi, Ningxia, Gansu, Shaanxi, Sichuan, Yunnan, Guizhou, and Taiwan. The Monolepta hieroglyphica (Motschulsky) belongs to an omnivorous pest, mainly damages crops such as beans, potatoes, alfalfa, corns, crowndaisy chrysanthemum, carrots, cruciferous vegetables, sunflowers, apricots, and apples. The larvae mainly feed on the roots of crops in the field and cannot damage the crops over the ground. From mid-July every year, adults can be found to damage leaves in corn and soybean fields. From the end of July to the beginning of August, a large number of adults mainly damage the filaments of corns and bite off the filaments of corns, which seriously affects pollination, causing sharp and spindle-shaped ears, and resulting in reduced corn production. Subsequently, the Monolepta hieroglyphica (Motschulsky) migrates to the soybean field to feed on soybean leaves or may migrate to the surrounding vegetable fields to damage vegetables. From 2009 to 2016, the damage area of the Monolepta hieroglyphica (Motschulsky) on corn increased from 16 million mu to nearly 40 million mu, and the occurrence area is doubled. In addition, the damaged areas also spread from the northwest China to major corn production areas such as the northeast China and north China.

The Chrysomelidae to which the Monolepta hieroglyphica (Motschulsky) belongs is the most diverse family in Coleoptera. Although the Monolepta hieroglyphica (Motschulsky) belongs to the same Chrysomelidae as potato beetles, except for the similarity in taxonomy, there are great differences in other morphological structures or habits. It just likes strawberries and apples in plants (both belong to Rosales rosaceae), they both have the characteristics of bisexual flowers, radial symmetry, and 5 petals and the like, but their fruits and plant shapes are very different. However, because people are less exposed to insects, especially agricultural pests, the less attention is paid to the differences in insect morphology, so people think that the morphology of insects is similar. In fact, the Monolepta hieroglyphica (Motschulsky) has unique morphological characteristics. For example, the adult body of the potato beetle is prominently raised, reddish-yellow, and glossy. The color of the elytra is slightly paler, and there are 5 black lengthwise strips on each elytra. There are large faint yellow spots on the two elytra of the adult of the Monolepta hieroglyphica (Motschulsky), respectively, the periphery of the spot is black, and half of the elytrum end is yellow.

The insects of the same Chrysomelidae are not only quite different in morphological characteristics, but also different in feeding habit. For example, a host of the potato beetle belonging to the same Chrysomelidae is mainly a solanaceae plant, and its adult feeds on the potato leaves and spawns between the potato leaves and leaf axils, and continuously damages the potato leaves after hatching. The potato beetle belongs to a life type all over the ground, and there is no migration of living space and no migration of food habits during this period. A host of the Monolepta hieroglyphica (Motschulsky) is relatively wide, including important crops such as beans, corns, potatoes, cotton, hemp, and vegetables. Adults feed on the parts of the corns over the ground spawn in the soil, and hatch in the next year after winter diapause. After hatching, larvae live in the soil and feed on the roots of the corns until eclosion. During this period, not only the living space is migrated, but also the food habits are changed. The difference in feeding habit also implies that enzymes and receptor proteins produced by a digestive system in a body are different. The enzymatic cleavage activation in insect gut, receptor binding on the insect gut and a physicochemical environment in the insect gut are key points for achieving the effect of a β-pore-forming protein. Only after the β-pore forming protein can be digested into active fragments and bound to the receptor on an epithelial cell membrane of the insect gut, it is possible to make a certain β-pore forming protein have an inhibitory activity aganist the pests. The receptor binding process requires accurate matching, and often a single amino acid difference in the pore forming protein or receptor protein can cause changes in binding to the same receptor. For example, after an aerolysin protein belonging to the same β-pore forming protein has qualitative changes in the virulence of a CTLL-2 cell line after R336A mutation (Osusky, Teschk et al, 2008). Likewise, since the receptor is changed, the virulence of the same β-pore forming protein may also be changed. For example, dsRNA is used to inhibit a HAVCR1 gene in a MDCK cell line, resulting in a hundred-fold difference in the virulence of an epsilon-toxin protein on cells (Ivie, Fennessey et al, 2011). The above fully indicates that the interaction between the β-pore forming protein and enzymes and receptors in insects is complex and unpredictable.

Callosobruchus chinensis (Linnaeus) in the present disclosure is a kind of stored product insects, which belongs to the same Coleoptera Chrysomelidae as the potato beetle. The pest mainly damages kidney beans, cowpeas, lentils, peas, broad beans, mung beans, red beans, and the like. Adults may spawn on beans in a warehouse or pods in the fields, and each female may spawn 70-80 eggs. The larvae bore into the pods and beans after hatching, and the adults have states of suspended animation. The pests may live through the winter in the beans at each insect stage and pupate and emerge in the following spring.

Henosepilachna vigintioctomaculata (Motschulsky) in the present disclosure belongs to Coleoptera Coccinellidae, which mainly damages potatoes. The food habits and living space of the pest are consistent with the potato beetles. Both adults feed on the potato leaves and spawn between the potato leaves and leaf axils, and the larvae continuously feed on the potato leaves after hatching.

The genome of the plant, plant tissue or plant cell in the present disclosure refers to any genetic materials in the plant, plant tissue or plant cell, and includes a cell nucleus and plastid and mitochondrial genome.

The polynucleotide and/or nucleotide described in the present disclosure forms a complete “gene” that encodes a protein or polypeptide in the required host cell. It is very easily recognized by those skilled in the art that the polynucleotide and/or nucleotide of the present disclosure may be placed under the control of a regulatory sequence in the target host.

It is well-known to those skilled in the art that DNA typically exists in a double-stranded form. In this arrangement, one strand is complementary to the other strand and vice versa. Other complementary strands of DNA are produced as a result of DNA replication in the plants. In this way, the present disclosure includes the use of the polynucleotide exemplified in a sequence listing and complementary strands thereof. A “coding strand” as commonly used in the field refers to a strand that binds to an antisense strand. In order to express a protein in vivo, typically one strand of DNA is transcribed into a complementary strand of mRNA, it serves as a template for translation of the protein. mRNA is actually transcribed from the “antisense” strand of DNA. The “sense” or “coding” strand has a series of codons (the codon is three nucleotides, and a specific amino acid may be produced by reading three at a time), and it may be read as an open reading frame (ORF) to form a target protein or peptide. The present disclosure also includes RNA that is functionally equivalent to the exemplified DNA.

The nucleic acid molecule or fragment thereof in the present disclosure hybridizes to the ACh1 gene of the present disclosure under stringent conditions. Any conventional nucleic acid hybridization or amplification methods may be used to identify the presence of the ACh1 gene of the present disclosure. The nucleic acid molecule or fragment thereof is capable of specifically hybridizing with other nucleic acid molecules under certain circumstances. In the present disclosure, if two nucleic acid molecules may form an anti-parallel double-stranded nucleic acid structure, it may be said that the two nucleic acid molecules may specifically hybridize with each other. If the two nucleic acid molecules show complete complementarity, one nucleic acid molecule is said to be a “complement” of the other nucleic acid molecule. In the present disclosure, while each nucleotide of one nucleic acid molecule is complementary to the corresponding nucleotide of the other nucleic acid molecule, the two nucleic acid molecules are said to show the “complete complementarity”. If the two nucleic acid molecules may hybridize to each other with sufficient stability such that they anneal and bind to each other under at least conventional “low stringency” conditions, the two nucleic acid molecules are said to be “minimally complementary”. Similarly, if the two nucleic acid molecules may hybridize to each other with the sufficient stability such that they anneal and bind to each other under conventional “high stringency” conditions, the two nucleic acid molecules are said to have “complementarity”. Deviation from the complete complementarity is permissible as long as such deviation does not completely prevent the two molecules from forming the double-stranded structure. In order for a nucleic acid molecule to function as a primer or a probe, it only needs to be sufficiently complementary in its sequence, as to allow for the formation of the stable double-stranded structure under adopted particular solvent and salt concentration.

In the present disclosure, the substantially homologous sequence is a section of a nucleic acid molecule, the nucleic acid molecule may specifically hybridize with a complementary strand of another matched nucleic acid molecule under highly stringent conditions. Suitable stringent conditions to promote the DNA hybridization, for example, treatment with 6.0× sodium chloride/sodium citrate (SSC) at about 45° C., and followed by washing with 2.0× SSC at 50° C., are well-known to those skilled in the art. For example, the salt concentration in a washing step may be selected from about 2.0×SSC and 50° C. under the low stringency conditions to about 0.2×SSC and 50° C. under the high stringency conditions. In addition, the temperature condition in the washing step may be increased from about 22° C. at a room temperature under the low stringency conditions to about 65° C. under the high stringency conditions. Both the temperature condition and the salt concentration may be changed, or one of which may be kept unchanged while the other variable is changed. Preferably, the stringency condition described in the present disclosure may be specific hybridization in 6×SSC and 0.5% sodium dodecyl sulfate (SDS) solutions at 65° C., and then membrane-washing once with 2×SSC, 0.1 % SDS and 1×SSC and 0.1 % SDS.

Therefore, sequences that have the insecticidal activity and hybridize to SEQ ID NO:5, SEQ ID NO:6, SEQ ID NO:7, or SEQ ID NO:8 of the present disclosure under the stringency condition are included in the present disclosure. These sequences have at least about 40%-50% of the identity with the sequences of the present disclosure, about 60%, 65% or 70% of the identity, even at least about 75%, 80%, 85%, 90%, 91%, 92%, 93%, 94%, 95%, 96%, 97%, 98%, 99% or greater sequence identity.

The genes and proteins described in the present disclosure include not only a specific exemplified sequence, but also include parts and/or fragments (including internal and/or terminal deletion as compared with a full-length protein) that preserve the characteristics of the insecticidal activity of the specific exemplified protein, a variant, a mutant, a substitute (a protein with a substituted amino acid), a chimera and a fusion protein. The “variant” or “variation” refers to a nucleotide sequence encoding the same protein or encoding an equivalent protein with the insecticidal activity. The “equivalent protein” refers to a protein that has the same or substantially the same biological activity against the Monolepta hieroglyphica (Motschulsky) as the claimed protein.

A “fragment” or “truncation” of the DNA molecule or protein sequence described in the present disclosure refers to a portion of the original DNA or protein sequence (nucleotide or amino acid) involved or an artificially modified form thereof (for example, a sequence suitable for plant expression), the length of the aforementioned sequence may have a change but is long enough to ensure that the (encoded) protein is an insect toxin.

A standard technology may be used to modify the gene and construct the genetic variant easily, for example, a technology for manufacturing a point mutation which is well-known in the field. As another example, U.S. Pat. No. 5605793 describes a method for producing additional molecular diversity using DNA reassembly after random fragmentation. The fragment of the full-length gene may be manufactured by using a commercial endonuclease, and an exonuclease may be used according to a standard procedure. For example, enzymes such as Bal31 or site-directed mutagenesis may be used to systematically excise nucleotides from the ends of these genes. The genes encoding the active fragments may also be obtained by using a plurality of restriction enzymes. The active fragments of these toxins may be obtained directly by using proteases.

The present disclosure may derive equivalent proteins and/or genes encoding these equivalent proteins from a β-pore forming protein isolate and/or a DNA library. There are various ways to obtain the insecticidal protein of the present disclosure. For example, antibodies of the insecticidal protein disclosed and claimed in the present disclosure may be used to identify and isolate other proteins from protein mixtures. In particular, the antibodies may arise from a portion of the protein that is most constant and most different from other β-pore forming proteins. These antibodies may then be used to specifically identify the equivalent proteins with the characteristic activity by immunoprecipitation, an enzyme-linked immunosorbent assay (ELISA), or a western blotting method. Antibodies of the proteins or the equivalent proteins or the fragments of such proteins disclosed in the present disclosure may be easily prepared by the standard procedure in the field. The genes encoding these proteins may then be obtained from the microorganisms.

Due to the redundancy of genetic codons, many different DNA sequences may encode the same amino acid sequence. The generation of these alternative DNA sequences encoding the same or substantially same protein is within the technological level of those skilled in the art. These various DNA sequences are included within a scope of the present disclosure. The “substantially same” sequence refers to a sequence with amino acid substitution, deletion, addition or insertion that does not substantially affect the insecticidal activity, and also includes a fragment that retains the insecticidal activity.

The substitution, deletion or addition of the amino acid sequence in the present disclosure is a routine technology in the field, preferably such an amino acid change is: a small property change, namely conservative amino acid substitution that does not significantly affect the folding and/or activity of the protein; small deletion, typically deletion of about 1-30 amino acids; small amino- or carboxy-terminal extension, for example, amino-terminal extension of one methionine residue; and a small linker peptide, for example, the length of about 20-25 residues.

Examples of the conservative substitution are those that occur within the following amino acid groups: basic amino acids (such as an arginine, a lysine, and a histidine), acidic amino acids (such as a glutamic acid and an aspartic acid), polar amino acids (such as a glutamine, and an asparagine), hydrophobic amino acids (such as a leucine, an isoleucine, and a valine), aromatic amino acids (such as a phenylalanine, a tryptophan, and a tyrosine), and small molecular amino acids (such as a glycine, an alanine, a serine, a threonine, and a methionine). Those amino acid substitutions that generally do not change the specific activity are well-known in the field, and already described, for example, by N. Neurath and R. L. Hill in “Protein” published by Academic Press, New York in 1979. The most common interchanges are Ala/Ser, Val/lle, Asp/Glu, Thu/Ser, Ala/Thr, Ser/Asn, Ala/Val, Ser/Gly, Tyr/Phe, Ala/Pro, Lys/Arg, Asp/Asn, Leu/Ile, Leu/Val, Ala/Glu and Asp/Gly, and their opposite interchanges.

It is apparent to those skilled in the art that such substitutions may occur outside areas important to the function of the molecule, and still produce the active polypeptide. The amino acid residues that are essential for the activity of the polypeptide of the present disclosure and are therefore selected not to be substituted may be identified according to methods known in the field, such as site-directed mutagenesis or alanine-scanning mutagenesis (referring to, for example, Cunningham and Wells, 1989, Science 244: 1081-1085). The latter technology is to introduce a mutation at each positively charged residue in the molecule, and to test the inhibitory activity of the mutant molecules obtained, thereby the amino acid residues that are important to the activity of the molecule are determined. Substrate-enzyme interaction sites may also be determined by analysis of its three-dimensional structure, this three-dimensional structure may be determined by technologies such as nuclear magnetic resonance analysis, crystallography, or photoaffinity labeling (referring to, for example, de Vos et al., 1992, Science 255:306-312; Smith et al., 1992, J. Mol. Biol 224:899-904; and Wlodaver et al., 1992, FEBS Letters 309:59-64).

In the present disclosure, the ACh1 protein includes, but is not limited to, SEQ ID NO:1, SEQ ID NO:2, SEQ ID NO:3, or SEQ ID NO:4, and amino acid sequences having certain identity with the amino acid sequence shown in SEQ ID NO:1, SEQ ID NO:2, SEQ ID NO:3, or SEQ ID NO:4 are also included in the present disclosure. These sequences are typically greater than 78% of similarity/identity of the sequence of the present disclosure, preferably greater than 85%, more preferably greater than 90%, even more preferably greater than 95%, and may be greater than 99%. Preferred polynucleotides and proteins of the present disclosure may also be defined according to more specific ranges of the identity and/or similarity. For example, there are 78%, 79%, 80%, 81%, 82%, 83%, 84%, 85%, 86%, 87%, 88%, 89%, 90%, 91%, 92%, 93%, 94%, 95%, 96%, 97%, 98% or 99% of the identity and/or similarity with the sequences exemplified in the present disclosure.

The regulatory sequence described in the present disclosure includes, but is not limited to, a promoter, a transit peptide, a terminator, an enhancer, a leader sequence, an intron, and other regulatory sequences operably linked to the ACh1 protein.

The promoter is a promoter expressible in the plant, and the “promoter expressible in the plant” refers to a promoter that ensures the expression of the coding sequence linked to it in plant cells. The promoter expressible in the plant may be a constitutive promoter. Examples of the promoter that direct the constitutive expression in the plant include, but are not limited to, a 35S promoter derived from a cauliflower mosaic virus, a maize Ubi promoter, a promoter of a rice GOS2 gene and the like. Alternatively, the promoter expressible in the plant may be a tissue-specific promoter, namely the promoter directs the expression of the coding sequence to a higher level in some tissues of the plant, such as in a green tissue, than in other tissues of the plant (may be determined by a conventional RNA test), such as a PEP carboxylase promoter. Alternatively, the promoter expressible in the plant may be a wound-inducible promoter. The wound-inducible promoter or a promoter that directs a wound-induced expression pattern means that the expression of the coding sequence under the control of the promoter is significantly increased while the plant is subjected to a mechanical or insect-induced wound compared to normal growth conditions. Examples of the wound-inducible promoter include, but are not limited to, promoters of protease inhibitory genes (pin I and pin II) of potato and tomato and a promoter of a maize protease inhibitor gene (MPI).Examples of the wound-inducible promoter include, but are not limited to, promoters of protease inhibitory genes (pin I and pin II) of potato and tomato and a promoter of a maize protease inhibitor gene (MPI).

The transit peptide (also known as a secretion signal sequence or a targeting sequence) directs a transgenic product to a specific organelle or cellular compartment, the transit peptide may be heterologous to the receptor protein, for example, by using a transit peptide sequence encoding a chloroplast to target the chloroplast, or using a ‘KDEL’ retention sequence to target an endoplasmic reticulum, or using CTPP of a barley lectin gene to target a vacuole.

The leader sequence includes, but is not limited to, a picornavirus leader sequence, such as an encephalomyocarditis virus 5′ non-coding region (EMCV) leader sequence; a potato Y virus group leader sequence, such as a maize dwarf mosaic virus (MDMV) leader sequence; a human immunoglobulin heavy chain binding protein (BiP); an untranslated leader sequence of coat protein mRNA of alfalfa mosaic virus (AMV RNA4); and a tobacco mosaic virus (TMV) leader sequence.

The enhancer includes, but is not limited to, a cauliflower mosaic virus (CaMV) enhancer, a figwort mosaic virus (FMV) enhancer, a carnation etched ring virus (CERV) enhancer, a cassava vein mosaic virus (CsVMV) enhancer, a mirabilis mosaic virus (MMV) enhancer, a cestrum yellow leaf curling virus (CmYLCV) enhancer, a cotton leaf curl multan virus (CLCuMV), a commellna yellow motlle virus (CoYMV) and a peanut chlorella leaf strip virus (PCLSV) enhancer.

For monocot applications, the intron includes, but is not limited to, a maize hsp70 intron, a maize ubiquitin intron, an Adh intron 1, a sucrose synthase intron, or a rice Act1 intron. For dicot applications, the intron includes, but is not limited to, a CAT-1 intron, a pKANNIBAL intron, a PIV2 intron, and a “super ubiquitin” intron.

The terminator may be a suitable polyadenylation signal sequence functioning in the plant, including, but not limited to, a polyadenylation signal sequence derived from a nopaline synthase (NOS) gene of Agrobacterium tumefaciens, a polyadenylation signal sequence derived from a protease inhibitor II (pin II) gene, a polyadenylation signal sequence derived from a pea ssRUBISCO E9 gene, and a polyadenylation signal sequence derived from a α-tubulin gene.

The “operably linked” in the present disclosure refers to association of nucleic acid sequences such that one sequence may provide a desired function for the linked sequence. In the present disclosure, the “operably linked” may be to link a promoter with an interested sequence, so that the transcription of the interested sequence is controlled and regulated by the promoter. While the interested sequence encodes a protein and the expression of the protein is desired, the “operably linked” means that: the promoter is linked to the sequence, so that an obtained transcript is efficiently translated in a linkage mode. If the linkage of the promoter to the coding sequence is transcript fusion and the expression of the encoded protein is desired, such linkage is manufactured, so that the first translation initiation codon in the obtained transcript is an initiation codon of the coding sequence. Alternatively, if the linkage of the promoter to the coding sequence is translational fusion and the expression of the encoded protein is desired, such linkage is manufactured, so that the first translation initiation codon contained in a 5′ untranslated sequence is linked to the promoter, and a relationship between an obtained translation product and a translational open reading frame encoding the desired protein accords with the reading frame in the linkage mode. The nucleic acid sequence that may be “operably linked” includes, but is not limited to: sequences providing gene expression functions (namely gene expression elements such as a promoter, a 5′ untranslated region, an intron, a protein coding region, a 3′ untranslated region, a poly adenylation site and/or a transcription terminator), sequences providing DNA transfer and/or integration functions (namely a T-DNA border sequence, a site-specific recombinase recognition site, and an integrase recognition site), sequences providing selectivity functions (namely an antibiotic resistance marker, and a biosynthetic gene), sequences providing scoreable marker functions, sequences that assist in sequence operation in vitro or in vivo (namely a polylinker sequence, and a site-specific recombination sequence) and sequences providing replication functions (namely a bacterial replication origin, a autonomously replicating sequence, and a centromeric sequence).

In the present disclosure, the “insecticide” or “insect resistance” means that it is toxic to crop pests, thereby the “control” and/or “prevention” of the crop pests is achieved. Preferably, the “insecticide” or “insect resistance” means that the crop pests are killed. More specifically, the target insect is the Monolepta hieroglyphica (Motschulsky).

The ACh1 protein in the present disclosure is virulent to the Monolepta hieroglyphica (Motschulsky). The plant in the present disclosure, especially the corn, the soybean and the cotton, contains an exogenous DNA in its genome. The exogenous DNA contains a nucleotide sequence encoding the ACh1 protein. The Monolepta hieroglyphica (Motschulsky) is in contact with the protein by ingesting the plant tissue, and after the contact, the growth of the Monolepta hieroglyphica (Motschulsky) is inhibited and/or death is caused. The inhibition means lethal or sub-lethal. At the same time, the plant should be morphologically normal, and may be cultivated under a conventional method for consumption and/or generation of products. In addition, the plant may substantially eliminate the need for a chemical or biological pesticide (the chemical or biological pesticide is an insecticide against the Monolepta hieroglyphica (Motschulsky) targeted by the ACh1 protein).

The expression level of an insecticidal protein in the plant material may be detected by a plurality of methods described in the field, for example, by applying a specific primer to quantify mRNA encoding the insecticidal protein produced in the tissue, or directly specifically detecting the amount of the insecticidal protein produced.

Different tests may be applied to determine the insecticidal effect of the insecticidal protein in the plant. In the present disclosure, the target insect is mainly the Monolepta hieroglyphica (Motschulsky).

In the present disclosure, the ACh1 protein may have an amino acid sequence shown in SEQ ID NO:1, SEQ ID NO:2, SEQ ID NO:3, or SEQ ID NO:4 in a sequence listing. In addition to the coding region containing the ACh1 protein, other elements may also be included, such as a protein encoding a selectable marker.

In addition, an expression cassette containing the polynucleotide sequence encoding the ACh1 protein of the present disclosure may also be expressed in the plant together with at least one protein encoding a herbicide resistance gene, the herbicide resistance gene includes, but not limited to, a glufosinate-ammonium resistance gene (such as a bar gene, and a pat gene), a Betanal resistance gene (such as a pmph gene), a glyphosate resistance gene (such as an EPSPS gene), a bromoxynil resistance gene, a sulfonylurea resistance gene, an anti-herbicide dalapon resistance gene, an anti-cyanamide resistance gene or a resistance gene of a glutamine synthase inhibitor (such as PPT), as to obtain the transgenic plant having both high insecticidal activity and herbicide resistance.

In the present disclosure, the exogenous DNA is introduced into the plant, for example, the gene or expression cassette or recombinant vector encoding the ACh1 protein is introduced into the plant cell, and the conventional transformation method includes, but not limited to, agrobacterium-mediated transformation, micro-emission bombardment, direct DNA ingestion into a protoplast, electroporation, or whisker silicon-mediated DNA introduction.

The present disclosure provides a use of an insecticidal protein and has the following advantages.

1. Prevention and treatment of internal causes: The prior arts mainly control the harm of the Monolepta hieroglyphica (Motschulsky) by the external action namely the external causes, for example, the agricultural control, the chemical control, the physical control and the biological control; and the present disclosure controls the Monolepta hieroglyphica (Motschulsky) by producing the ACh1 protein that may kill the Monolepta hieroglyphica (Motschulsky) in the plant, namely the Monolepta hieroglyphica (Motschulsky) is controlled by the internal causes.

2. No pollution and no residue: Although the chemical control method used in the prior art plays a certain role in controlling the harm of the Monolepta hieroglyphica (Motschulsky), it also brings the pollution, damage and residue to humans, livestocks and farmland ecosystems; and using the method of the present disclosure to control the Monolepta hieroglyphica (Motschulsky), the above adverse consequences may be eliminated.

3. Prevention and control during whole growth period: The methods used in the prior arts to control the Monolepta hieroglyphica (Motschulsky) are all by stages, and the present disclosure is to protect the plant during the whole growth period, and the transgenic plant (ACh1 protein) may be prevented from being attacked by the Monolepta hieroglyphica (Motschulsky) from germination, growth, to flowering and fruiting.

4. Whole plant control: Most of the methods used in the prior art to control the Monolepta hieroglyphica (Motschulsky) are localized, such as foliar spraying; and the present disclosure protects the entire plant, for example, roots, leaves, stems, fruits, tassels, female ears, anthers or filaments of the transgenic plant (ACh1 protein) are all resistant to the attack of the Monolepta hieroglyphica (Motschulsky).

5. Stable effect: Whether it is the agricultural control method or the physical control method used in the prior art, it is necessary to use the environmental conditions to control the pests, and there are many variable factors; the present disclosure is to express the ACh1 protein in the plant, which effectively overcomes the disadvantages of the unstable environmental conditions, and the control effect of the transgenic plant (ACh1 protein) of the present disclosure is stable and consistent in different places, different times and different genetic backgrounds.

6. Simpleness, convenience and economy: The present disclosure only needs to plant the transgenic plant capable of expressing the ACh1 protein, and does not need to adopt other measures, thereby reducing a lot of manpower, material resources and financial resources.

7. Complete effect: The methods used in the prior art to control the Monolepta hieroglyphica (Motschulsky) are not thorough in effect, and only play a role in relieving; and the transgenic plant (ACh1 protein) of the present disclosure may cause a large number of deaths of the newly hatched larvae of the Monolepta hieroglyphica (Motschulsky).

The technical schemes of the present disclosure are further described in detail below by drawings and embodiments.

### BRIEF DESCRIPTION OF THE DRAWINGS

FIG. 1 is a construction flowchart of a recombinant expression vector DBN01-P containing an ACh1 nucleotide sequence for the use of the insecticidal protein according to the present disclosure.

FIG. 2 is a construction flowchart of a recombinant expression vector DBN01-T containing an ACh1 nucleotide sequence for the use of the insecticidal protein according to the present disclosure.

FIG. 3 is a construction flowchart of a plant recombinant expression vector DBN01-B containing an ACh1 nucleotide sequence for the use of the insecticidal protein according to the present disclosure.

### DETAILED DESCRIPTION OF THE EMBODIMENTS

The technical schemes of the use of the insecticidal protein of the present disclosure are further described below by specific embodiments.

### First Embodiment: Acquisition and Synthesis of Gene

### 1. Acquisition of the Nucleotide Sequence

An amino acid sequence of an ACh1_1 insecticidal protein (309 amino acids) is shown in SEQ ID NO:1 in a sequence listing. An ACh1_1 bacterial nucleotide sequence (930 nucleotides) encoding the amino acid sequence corresponding to the ACh1_1 insecticidal protein in bacteria is shown in SEQ ID NO: 5 in the sequence listing. An ACh1_1 plant nucleotide sequence (930 nucleotides) encoding the amino acid sequence corresponding to the ACh1_1 insecticidal protein is shown in SEQ ID NO:9 in the sequence listing.

An amino acid sequence of an ACh1_2 insecticidal protein (306 amino acids) is shown in SEQ ID NO:2 in a sequence listing. An ACh1_2 bacterial nucleotide sequence (921 nucleotides) encoding the amino acid sequence corresponding to the ACh1_2 insecticidal protein in bacteria is shown in SEQ ID NO: 6 in the sequence listing. An ACh1_2 plant nucleotide sequence (921 nucleotides) encoding the amino acid sequence corresponding to the ACh1_2 insecticidal protein is shown in SEQ ID NO:10 in the sequence listing.

An amino acid sequence of an ACh1_3 insecticidal protein (309 amino acids) is shown in SEQ ID NO:3 in a sequence listing. An ACh1_3 bacterial nucleotide sequence (930 nucleotides) encoding the amino acid sequence corresponding to the ACh1_3 insecticidal protein in bacteria is shown in SEQ ID NO: 7 in the sequence listing. An ACh1_3 plant nucleotide sequence (930 nucleotides) encoding the amino acid sequence corresponding to the ACh1_3 insecticidal protein is shown in SEQ ID NO:11 in the sequence listing.

An amino acid sequence of an ACh1_4 insecticidal protein (309 amino acids) is shown in SEQ ID NO:4 in a sequence listing. An ACh1_4 bacterial nucleotide sequence (930 nucleotides) encoding the amino acid sequence corresponding to the ACh1_4 insecticidal protein in bacteria is shown in SEQ ID NO: 8 in the sequence listing. An ACh1_4 plant nucleotide sequence (930 nucleotides) encoding the amino acid sequence corresponding to the ACh1_4 insecticidal protein is shown in SEQ ID NO:12 in the sequence listing.

### 2. Synthesis of Above Nucleotide Sequence

The bacterial nucleotide sequences (as shown in SEQ ID NO:5 to SEQ ID NO:8 in the sequence listing) of ACh1_1, ACh1_2, ACh1_3, and ACh1_4 and the plant nucleotide sequence (as shown in SEQ ID NO:9) of ACh1_1 are synthesized.

### Second Embodiment: Construction of Recombinant Expression Vector and Transformation Of Recombinant Expression Vector into Escherichia Coli to Obtain ACh1 Protein

### 1. Construction of Recombinant Expression Vector Containing ACh1 Gene

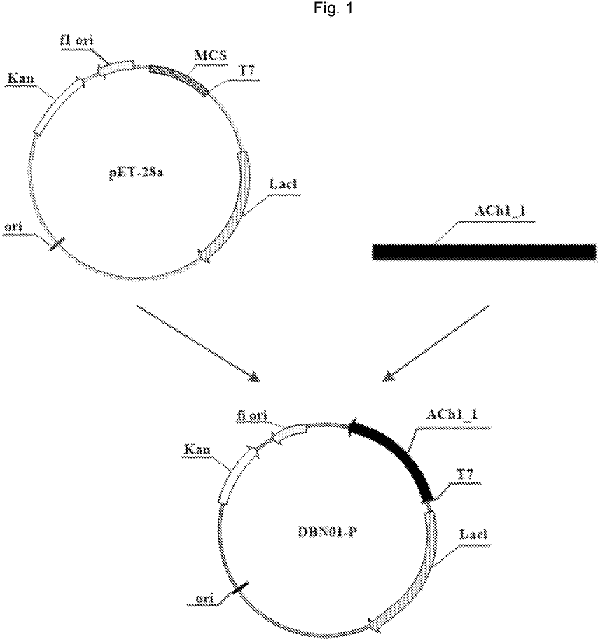

The ACh1_1 bacterial nucleotide sequence synthesized in the first embodiment is linked into a protein expression vector pET28a (Novagen, USA, CAT: 69864-3); operation steps are performed according to the specification of the product pET28a vector of Novagen, so as to obtain a recombinant expression vector DBN01-P; and a construction flow is shown in FIG. 1 (where Kan represents a kanamycin resistance gene, f1 ori represents the origin of replication of phage f1, Lacl is a Lacl initiation codon, ACh1_1 is an ACh1_1 bacterial nucleotide sequence (SEQ ID NO:5), and MCS represents multiple cloning sites).

According to the above method for constructing the recombinant expression vector DBN01-P, the synthesized ACh1_2 bacterial nucleotide sequence is linked to the protein expression vector pET28a, so as to obtain a recombinant expression vector DBN02-P, and ACh1_2 is the ACh1_2 bacterial nucleotide sequence (SEQ ID NO:6).

According to the above method for constructing the recombinant expression vector DBN01-P, the synthesized ACh1_3 bacterial nucleotide sequence is linked to the protein expression vector pET28a, so as to obtain a recombinant expression vector DBN03-P, and ACh1_3 is the ACh1_3 bacterial nucleotide sequence (SEQ ID NO:7).

According to the above method for constructing the recombinant expression vector DBN01-P, the synthesized ACh1_4 bacterial nucleotide sequence is linked to the protein expression vector pET28a, so as to obtain a recombinant expression vector DBN04-P, and ACh1_4 is the ACh1_4 bacterial nucleotide sequence (SEQ ID NO:8).

### 2. Transformation of Recombinant Expression Vector Into Escherichia Coli to Obtain ACh1 Protein

Then, the recombinant expression vectors DBN01-P, DBN02-P, DBN03-P, and DBN04-P are transformed into Escherichia coli BL21(DE3) competent cells (Transgen, China, CAT: CD501) by a heat shock method; a positive colony is picked and placed in an LB liquid medium (10 g/L of a tryptone, 5 g/L of a yeast extract, 10 g/L of NaCl, 100 mg/L of an ampicillin, and pH is adjusted to 7.5 with NaOH); and culture is performed for 16h at 37° C. and at 200 r/min. The culture solution is then transferred to an YT culture medium according to the proportion of 1:10; and culture is performed at 37° C. and at 200 r/min. When an OD=600 value of the culture solution reaches 0.6-0.8, IPTG is added until a final concentration is 0.5 mM, so as to perform inducible expression for 6h, and the culture solution is centrifuged to collect the cells; the supernatant is discarded, resuspending is performed after PBS is added, and then ultrasonic disruption is performed; and the expression protein is detected by SDS-PAGE, the protein concentration is estimated, and preservation is performed at -20° C. for later use.

### Third Embodiment, Identification of Inhibitory Activity Against Monolepta Hieroglyphica (Motschulsky) by Feeding ACh1 Protein

Inhibitory activity against the Monolepta hieroglyphica (Motschulsky), the Callosobruchus chinensis (Linnaeus) and the Henosepilachna vigintioctomaculata (Motschulsky) is detected by using the ACh1_1, ACh1_2, ACh1_3, and ACh1_4 proteins obtained in 2 in Embodiment II. A total of 4 treatments are designed for each pest, which respectively are ACh1_1, ACh1_2, ACh1_3, and ACh1_4; and 1 negative control treatment is designed, which is GFP.

Monolepta hieroglyphica (Motschulsky): protein liquid of ACh1_1, ACh1_2, ACh1_3, ACh1_4, and GFP are respectively mixed in feed, and a final concentration is 50 µg/g. Each group of treatments is repeated for 3 times.

Callosobruchus chinensis (Linnaeus): mung beans are immersed in the protein liquid of ACh1_1, ACh1_2, ACh1_3, ACh1_4, and GFP according to the concentration of 50 µg/g. Each group of treatments is repeated for 3 times.

Henosepilachna vigintioctomaculata (Motschulsky): potato leaves are immersed in the protein liquid of ACh1_1, ACh1_2, ACh1_3, ACh1_4, and GFP according to the concentration of 50 µg/g. Each group of treatments is repeated for 3 times.

TABLE 1

Results of Monolepta hieroglyphica (Motschulsky), the Callosobruchus chinensis (Linnaeus) and the Henosepilachna vigintioctomaculata (Motschulsky) by feeding ACh1 protein
Test insect

| | | | |
|---|---|---|---|
| Serial number of proteins | Monolepta hieroglyphica (Motschulsky) | Callosobruchus chinensis (Linnaeus) | Henosepilachna vigintioctomaculata (Motschulsky) |
| ACh1_1 | + | - | - |
| ACh1_2 | + | - | - |
| ACh1_3 | + | - | - |
| ACh1_4 | + | - | - |
| GFP | - | - | - |

“+” means that there is an inhibitory activity against the pest; and “-” means that there is no inhibitory activity against the pest

Results of Table 1 show that, the ACh1_1, ACh1_2, ACh1_3, and ACh1_4 proteins have desirable inhibitory activity against the Monolepta hieroglyphica (Motschulsky), and have no inhibitory activity against on the Callosobruchus chinensis (Linnaeus) (same family) and the Henosepilachna vigintioctomaculata (Motschulsky) that belong to the same Coleoptera.

The above results fully indicate that the toxicity of the insecticidal toxin protein to insects is not necessarily related to the family of insects, but is inseparable from the mechanism of action of the insecticidal protein. That is to say, the enzymatic cleavage activation in insect gut, receptor binding on the insect gut and a physicochemical environment in the insect gut are key points for achieving the effect of a β-pore forming toxin, and the interaction between the β-pore forming protein and enzymes and receptors in insects is complex and unpredictable.

### Fourth Embodiment, Construction of Plant Expression Vector

### 1. Construction of Recombinant Cloning Vector Containing ACh1 Gene

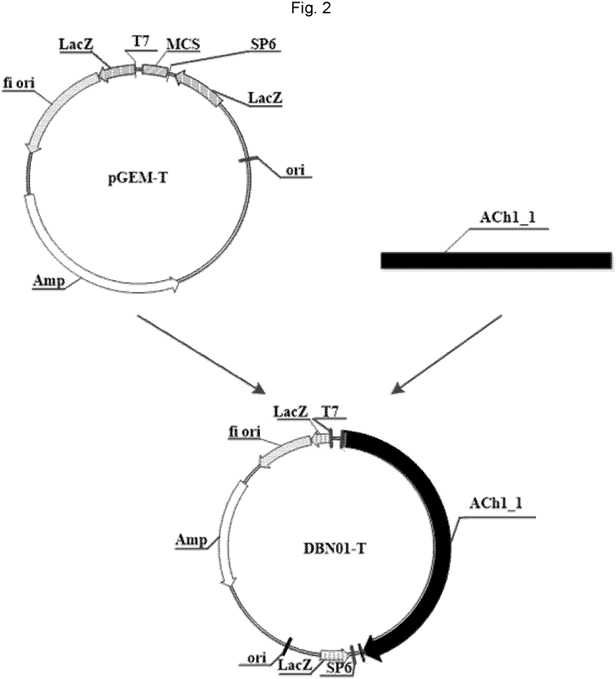

The synthesized ACh1_1 plant nucleotide sequence is linked to a cloning vector pGEM-T (Promega, Madison, USA, CAT: A3600), and an operation step is performed according to instructions of a pGEM-T vector product of Promega Company, to obtain a recombinant cloning vector DBN01-T, and its construction process is shown in FIG. 2 (herein, Amp represents an ampicillin resistance gene; flori represents the origin of replication of phage f1; LacZ is an LacZ initiation codon; SP6 is an SP6 RNA polymerase promoter; T7 is a T7 RNA polymerase promoter; ACh1_1 is the ACh1_1 plant nucleotide sequence (SEQ ID NO:9); and MCS represents multiple cloning sites).

Then, the recombinant cloning vector DBN01-T is transformed into Escherichia coli T1 competent cells (Transgen, Beijing, China, CAT: CD501) by a heat shock method, and a white bacterial colony is picked, and placed in a Luria-Bertani (LB) liquid medium (10 g/L of a tryptone, 5 g/L of a yeast extract, 10 g/L of NaCl, 100 mg/L of an ampicillin, and pH is adjusted to 7.5 with NaOH) and cultured overnight at 37° C. Plasmids thereof are extracted by an alkaline method and stored at -20° C. for future use.

After the extracted plasmid is identified by enzyme digestion, the positive colonies are sequenced and verified, and results show that the ACh1_1 nucleotide sequence inserted in the recombinant cloning vector DBN01-T is the nucleotide sequence shown in the sequence listing (SEQ ID NO:9). That is to say, the ACh1_1 plant nucleotide sequence is correctly inserted.

### 2. Construction of the Recombinant Expression Vector Containing the ACh1 Gene

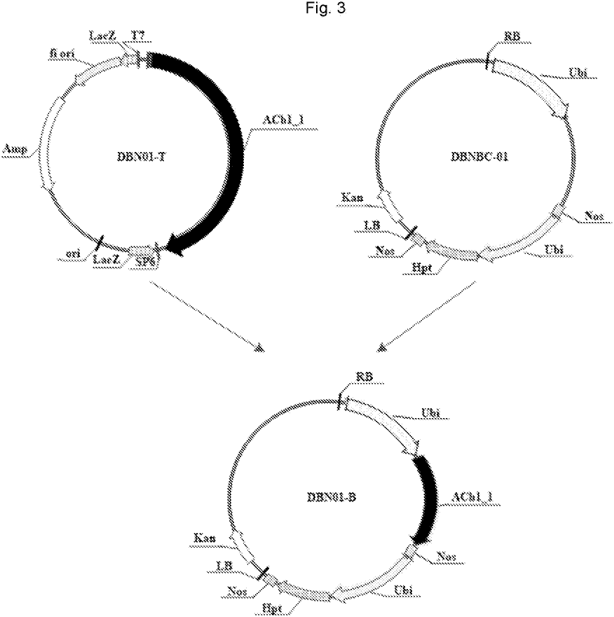

The recombinant cloning vector DBN01-T and the expression vector DBNBC-01 (vector framework: pCAMBIA2301 (provided by the CAMBIA institution)) are digested with restriction endonucleases, and an excised ACh1_1 plant nucleotide sequence fragment is inserted between the restriction endonuclease sites of the expression vector DBNBC-01. It is well-known to those skilled in the art to construct a vector with a conventional enzyme digestion method, the recombinant expression vector DBN01-B is constructed, and the construction flow is shown in FIG. 3 (Kan: kanamycin gene; RB: right border; prUbi: maize ubiquitin gene promoter (SEQ ID NO:15); ACh1_1: ACh1_1 plant nucleotide sequence (SEQ ID NO:9); tNos: terminator of nopaline synthase gene (SEQ ID NO:16); Hpt: hygromycin phosphotransferase gene (SEQ ID NO:17); and LB: left border).

The recombinant expression vector DBN01-B is transformed into the Escherichia coli T1 competent cells with the heat shock method; the white colony is picked and placed in the LB liquid medium (10 g/L of the tryptone, 5 g/L of the yeast extract, 10 g/L of NaCl, 50 mg/L of the kanamycin, and pH is adjusted to 7.5 with NaOH); and culture is performed overnight at 37° C., and plasmids thereof are extracted by an alkaline method. The extracted plasmid is identified by the restriction endonuclease digestion, and the positive colonies are sequenced and identified. The results show that the nucleotide sequence in the recombinant expression vector DBN01-B is the nucleotide sequence shown in SEQ ID NO:9 in the sequence listing, that is, the ACh1_1 plant nucleotide sequence.

### 3. Transformation of the Recombinant Expression Vector Into an Agrobacterium

The correctly constructed recombinant expression vector DBN01-B is transformed into agrobacterium LBA4404 (Invitrgen, Chicago, USA, CAT: 18313-015) through a liquid nitrogen method, and the transformation conditions are as follows: 100 µl of the agrobacterium LBA4404, and 3 µl of a plasmid DNA (the recombinant expression vector); it is placed in liquid nitrogen for 10 minutes, and a warm water bath is performed at 37° C. for 10 minutes; the transformed agrobacterium LBA4404 is inoculated in an LB tube, cultured for 2 hours under conditions of a temperature of 28° C. and a rotation speed of 200 rpm, and spread on an LB plate containing 50 mg/L of rifampicin and 100 mg/L of kanamycin until positive monoclones grow, the monoclones are picked for culture and plasmids thereof are extracted, the restriction endonuclease is used to verify the recombinant expression vector DBN01-B after being enzyme-digested, and results show that the structure of the recombinant expression vector DBN01-B is completely correct.

### Fifth Embodiment, Obtaining of Transgenic Corn Plant

According to the conventional agrobacterium infection method, the immature embryos of the aseptically cultured maize variety Zong 31 (Z31) are co-cultured with the agrobacterium transformed with the recombinant expression vector described in step 3 in the fourth embodiment, to transfer the T-DNA (including the promoter sequence of maize ubiquitin gene, the ACh1_1 nucleotide sequence, the Hpt gene and the Nos terminator sequence) in the recombinant expression vector DBN01-B constructed in step 2 in the fourth embodiment into a maize genome, so as to obtain a corn plant transformed with the ACh1_1 nucleotide sequence. In addition, a wild corn plant is used as a control.

For agrobacterium-mediated transformation of corns, briefly, immature embryos are isolated from the corns and are in contact with agrobacterium suspension. The agrobacterium can deliver the ACh1_1 nucleotide sequence to at least one cell (step 1: infection step) of one of the embryos. In this step, the embryos are preferably immersed in the agrobacterium suspension (OD₆₆₀=0.4-0.6, an infection medium (4.3 g/L of MS salt, MS vitamins, 300 mg/L of casein, 68.5 g/L of sucrose, 36 g/L of glucose, 40 mg/L of Acetosyringone (AS), and 1 mg/L of 2,4-dichlorophenoxyacetic acid (2,4-D), pH 5.3) to initiate inoculation. The embryos are co-cultured with the agrobacterium for a period of time (3 days) (Step 2: co-culture step). Preferably, the embryos are cultured in a solid culture medium (4.3 g/L of the MS salt, the MS vitamins, 300 mg/L of casein, 20 g/L of sucrose, 10 g/L of glucose, 100 mg/L of AS, 1 mg/L of 2,4-D, and 8 g/L of agar, pH5.8) after the infection step. After this co-culture phase, there may be an optional “recovery” step. In the “recovery” step, in a recovery culture medium (4.3 g/L of the MS salt, the MS vitamins, 300 mg/L of casein, 30 g/L of sucrose, 1 mg/L of 2,4-D, and 8 g/L of agar, pH5.8), there is at least one antibiotic (cephalosporin) known to inhibit the growth of the agrobacterium, and a selective agent for a plant transformant (Step 3: recovery step) is not added. Preferably, the embryos are cultured on a solid medium with the antibiotic without the selective agent, as to eliminate the agrobacterium and provide a recovery period for infected cells. Next, the inoculated embryos are grown on a culture medium containing the selective agent (hygromycin) and a grown transformed callus is selected (Step 4: selection step). Preferably, the embryos are cultured in the solid culture medium (4.3 g/L of the MS salt, the MS vitamins, 300 mg/L of casein, 5 g/L of sucrose, 50 mg/L of hygromycin, 1 mg/L of 2,4-D, and 8 g/L of agar, pH5.8) containing the selective agent, so as to cause the transformed cells to selectively grow. The callus are then regenerated into plants (Step 5: regeneration step), preferably, the callus grown on the medium containing the selective agent is cultured on the solid medium (MS differentiation medium and MS rooting medium) to regenerate the plant.

The screened resistant callus are transferred to the MS differentiation medium (4.3 g/L of the MS salt, the MS vitamins, 300 mg/L of casein, 30 g/L of sucrose, 2 mg/L of 6-benzyladenine, 50 mg/L of hygromycin, and 8 g/L of agar, pH5.8), and culture differentiation is performed at 25° C. The differentiated seedling is transferred to the MS rooting medium (2.15 g/L of the MS salt, the MS vitamins, 300 mg/L of casein, 30 g/L of sucrose, 1 mg/L of indole-3-acetic acid, and 8 g/L of agar, pH5.8); and the seedling is cultured to a height of about 10 cm at 25° C., and then moved to a greenhouse to grow until fruiting. In the greenhouse, culture is performed for 16h at 28° C. every day, and then culture is performed for 8 h at 20° C.

### Sixth Embodiment: Verification of Transgenic Maize Plants With TaqMan

About 100 mg of leaves of the corn plant transformed with the ACh1_1 plant nucleotide sequence is taken as a sample, and the genome DNA is extracted with DNeasy Plant Maxi Kit of Qiagen, and the copy number of the Hpt gene is detected by a Taqman probe fluorescence quantitative PCR method to determine the copy number of the ACh1_1 gene. At the same time, the wild corn plant is used as a control, and the detection and analysis are performed according to the above method. The experiment is repeated for 3 times, and the average value is taken.

A specific method to detect the copy number of the Hpt gene is as follows.

Step 11, 100 mg of the leaves of the corn plant transformed with the ACh1_1 nucleotide sequence and the wild corn plant are taken respectively, and ground into uniform slurry with liquid nitrogen in a mortar, and 3 replicates for each sample are taken.

Step 12, Qiagen’s DNeasy Plant Mini Kit is used to extract the genome DNA of the above samples, and a specific method refers to its product specification.

Step 13, NanoDrop 2000 (Thermo Scientific) is used to measure the genome DNA concentration of the above samples.

Step 14, the genome DNA concentration of the above samples is adjusted to the same concentration value, and the range of the concentration value is 80-100 ng/µl.

Step 15, the Taqman probe fluorescence quantitative PCR method is used to identify the copy number of the sample, the sample with the known copy number after the identification is used as a standard substance, and the sample of the wild corn plant is used as a control, 3 replicates for each sample are taken, and its average value is taken; and fluorescence quantitative PCR primer and probe sequences are as follows.

The following primers and probes are used to detect the Hpt nucleotide sequence.

Primer 1: cagggtgtcacgttgcaaga is as shown in SEQ ID NO:18 in the sequence listing.

Primer 2: ccgctcgtctggctaagatc is as shown in SEQ ID NO:19 in the sequence listing.

Probe 1: tgcctgaaaccgaactgcccgctg is as shown in SEQ ID NO:20 in the sequence listing.

A PCR reaction system is as follows.

| | |
|---|---|
| JumpStart™ Taq ReadyMix™ (Sigma) | 10 µl |
| 50× primer/probe mixture | 1 µl |
| Genomic DNA | 3 µl |
| Water (ddH₂O) | 6 µl |

The 50× primer/probe mixture contains 45 µl of each primer at a concentration of 1 mM, 50 µl of the probe at a concentration of 100 µM and 860 µl of 1×TE buffer, and is stored in a centrifuge tube at 4° C.

PCR reaction conditions are as follows.

| Step | Temperature | Time |
|---|---|---|
| 21 | 95° C. | 5 min |
| 22 | 95° C. | 30 s |
| 23 | 60° C. | 1 min |
| 24 | Returning to Step 22, and repeating for 40 times | |

Data is analyzed with SDS 2.3 software (Applied Biosystems).

The experimental results by analyzing the copy number of the Hpt genes show that, the ACh1_1 nucleotide sequence has been integrated into the genome of the tested corn plants, and the corn plants transformed with the ACh1_1 nucleotide sequence all obtain single-copy transgenic corn plants.

### Seventh Embodiment, Identification of Inhibitory Activity of Transgenic Corn Plants

The corn plant transformed with the ACh1_1 nucleotide sequence, the corresponding wild corn plant, and the corn plant identified as non-transgenic by Taqman are detected for the inhibitory activity against the Monolepta hieroglyphica (Motschulsky).

Fresh leaves (heart leaves) of the corn plant transformed with the ACh1_1 nucleotide sequence, the wild corn plant, and the corn plant (Stage V3-V4) identified as non-transgenic by Taqman are taken respectively, washed with sterile water and dried with gauze; then, the veins are removed from the corn leaves, the leaves are cut into strips of about 1 cm × 2 cm, and 1 piece of the cut strip-like leaf is taken and put the leaf on a moisturizing filter paper at the bottom of a circular plastic petri dish; 10 Monolepta hieroglyphica (Motschulsky) (larvae) are put in each petri dish; after the insect-testing petri dish is covered, the petri dish is put for 1 day under the conditions of a temperature of 24±2° C., a relative humidity of 70%-80%, and a photoperiod (light/dark) of 24:0; from the second day after infestation, the positive leaves are replaced every 2 days until the end of the experiment at Day 10 to test whether there was a significant difference in survival rate. A total of 3 lines are transformed into ACh1_1 nucleotide sequence, 1 line is identified as non-transgenic (NGM) by Taqman, and 1 line is identified as wild (CK). 5 plants are selected from each line for test, and each plant is tested repeatedly for 3 times. Results are shown in Table 2.

TABLE 2

Inhibitory activity experimental results of transgenic corn plants inoculated with Monolepta hieroglyphica (Motschulsky)

| | Test insect |
|---|---|
| Serial number of proteins | Monolepta hieroglyphica (Motschulsky) |
| ACh1_1 | + |
| NGM | - |
| CK | - |

“+” means that there is an inhibitory activity against pest; and “-” means that there is no inhibitory activity against pest

The results show that the corn plants transformed with the ACh1_1 nucleotide sequence have a desirable lethal effect on the Monolepta hieroglyphica (Motschulsky). Therefore, it indicates that the ACh1_1 protein shows resistance activity against the Monolepta hieroglyphica (Motschulsky) both in bacteria and in plants, and this activity is sufficient to have adverse effects on the growth of the Monolepta hieroglyphica (Motschulsky), so that the Monolepta hieroglyphica (Motschulsky) can be controlled in the fields. In addition, it is also possible to reduce the occurrence of diseases on the transgenic ACh1 plants by controlling the damage of the Monolepta hieroglyphica (Motschulsky), thereby greatly improving the yield and quality of the transgenic ACh1 plants.

In conclusion, through the use of the insecticidal protein of the present disclosure, ACh1 protein that can kill the Monolepta hieroglyphica (Motschulsky) is produced in bacteria and/or a plant body to control the Monolepta hieroglyphica (Motschulsky). Compared with an agricultural control method, a chemical control method, a physical control method and a biological control method used in the prior art, the present disclosure achieves the protection of whole growth period and whole plant on the plants so as to control the infestation of the Monolepta hieroglyphica (Motschulsky), and is pollution-free, residue-free, stable in effect, thorough, simple, convenient and economical.

Finally, it should be noted that the above embodiments are only used to illustrate the technical schemes of the present disclosure and not to limit them. Although the present disclosure is described in detail with reference to the preferred embodiments, those of ordinary skill in the art should understand that the technical schemes of the present disclosure may be modified or equivalently replaced without departing from the spirit and scope of the technical schemes of the present disclosure.

What is claimed is:

1. A method for controlling Monolepta hieroglyphica (Motschulsky), comprising allowing the Monolepta hieroglyphica (Motschulsky) to be at least in contact with an ACh1 protein;
preferably, the ACh1 protein is present in a host cell that produces at least the ACh1 protein, and the Monolepta hieroglyphica (Motschulsky) is in contact with at least the ACh1 protein by ingesting the host cell; and
more preferably, the ACh1 protein is present in at least a bacterium or a transgenic plant that generates the ACh1 protein, the Monolepta hieroglyphica (Motschulsky) is in contact with at least the ACh1 protein by ingesting the bacterium or a tissue of the transgenic plant, and after contacting, the growth of the Monolepta hieroglyphica (Motschulsky) is inhibited and/or death is caused, so as to achieve the control of the damage of the Monolepta hieroglyphica (Motschulsky) to plants.

2. The method for controlling Monolepta hieroglyphica (Motschulsky) according to claim 1, wherein the transgenic plant is soybean, wheat, barley, corns, tobacco, rice, rape, cotton, or sunflowers.

3. The method for controlling Monolepta hieroglyphica (Motschulsky) according to claim 1, wherein the tissue of the transgenic plant is a root, a leaf, a stem, a tassel, an ear, an anther, or a filament.

4. The method for controlling Monolepta hieroglyphica (Motschulsky) according to claim 1, wherein the ACh1 protein is an ACh1_1 protein, an ACh1_2 protein, an ACh1_3 protein, or an ACh1_4 protein.

5. The method for controlling Monolepta hieroglyphica (Motschulsky) according to claim 4, wherein the ACh1 protein has an amino acid sequence shown in SEQ ID NO:1, SEQ ID NO:2, SEQ ID NO:3, or SEQ ID NO:4.

6. The method for controlling Monolepta hieroglyphica (Motschulsky) according to claim 4, wherein a nucleotide sequence of the ACh1 protein in the bacteria is shown in SEQ ID NO:5, SEQ ID NO:6, SEQ ID NO:7, or SEQ ID NO:8; and a nucleotide sequence of the ACh1 protein in the transgenic plant is shown in SEQ ID NO:9, SEQ ID NO:10, SEQ ID NO:11, or SEQ ID NO:12.

7. The method for controlling Monolepta hieroglyphica (Motschulsky) according to claim 1, wherein the transgenic plant further comprises at least a second nucleotide different from the nucleotide encoding the ACh1 protein.

8. The method for controlling Monolepta hieroglyphica (Motschulsky) according to claim 7, wherein the second nucleotide encodes a Cry-like insecticidal protein, a Vip-like insecticidal protein, a protease inhibitor, lectin, α-amylase, or a peroxidase.

9. The method for controlling Monolepta hieroglyphica (Motschulsky) according to claim 8, wherein the second nucleotide encodes a Cry3Bb protein.

10. The method for controlling Monolepta hieroglyphica (Motschulsky) according to claim 9, wherein the Cry3Bb protein has an amino acid sequence shown in SEQ ID NO:13.

11. The method for controlling Monolepta hieroglyphica (Motschulsky) according to claim 10, wherein the second nucleotide has a nucleotide sequence shown in SEQ ID NO:14.

12. The method for controlling Monolepta hieroglyphica (Motschulsky) according to claim 7, wherein the second nucleotide is a dsRNA that inhibits an important gene in a target insect pest.

13. A method of producing a plant for controlling Monolepta hieroglyphica (Motschulsky), comprising introducing a polynucleotide sequence encoding an ACh1 protein into a genome of the plant.

14. The method of producing a plant for controlling Monolepta hieroglyphica (Motschulsky) according to claim 13, wherein the polynucleotide sequence of the ACh1 protein is shown in SEQ ID NO:9, SEQ ID NO:10, SEQ ID NO:11, or SEQ ID NO:12.

15. The method of producing a plant for controlling Monolepta hieroglyphica (Motschulsky) according to claim 13, wherein the ACh1 protein has an amino acid sequence shown in SEQ ID NO:1, SEQ ID NO:2, SEQ ID NO:3, or SEQ ID NO:4.

16. A method of producing a plant seed for controlling Monolepta hieroglyphica (Motschulsky), comprising hybridizing a plant obtained by the method according to claim 13 with a second plant, so as to produce a seed containing a polynucleotide sequence encoding an ACh1 protein.

17. A method of cultivating a plant for controlling Monolepta hieroglyphica (Motschulsky), comprising:
planting at least one plant seed, wherein the genome of the plant seed comprises a polynucleotide sequence encoding an ACh1 protein;
growing the plant seed into a plant; and
growing the plant under conditions that the Monolepta hieroglyphica (Motschulsky) is artificially inoculated and/or the hazard of the Monolepta hieroglyphica (Motschulsky) naturally occurs, and harvesting a plant that has an attenuated plant damage and/or has an increased plant yield compared with other plants that do not have the polynucleotide sequences encoding the ACh1 protein.

18. The method of cultivating a plant for controlling Monolepta hieroglyphica (Motschulsky) according to claim 17, wherein the polynucleotide sequence of the ACh1 protein is shown in SEQ ID NO:9, SEQ ID NO:10, SEQ ID NO:11, or SEQ ID NO:12.

19. The method of cultivating a plant for controlling Monolepta hieroglyphica (Motschulsky) according to claim 17, wherein the ACh1 protein has an amino acid sequence shown in SEQ ID NO:1, SEQ ID NO:2, SEQ ID NO:3, or SEQ ID NO:4.


SEQUENCE LISTING (20 sequences)

SEQ ID NO: 1 - protein, 309 aa, synthetic construct
MIFLAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 2 - protein, 306 aa, synthetic construct
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTSQEASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVVSQVSAGVYATVRFDQYDIHNLWTIEKTWYARHATLHNGKKISINNVTEMAPTSPIKTN

SEQ ID NO: 3 - protein, 309 aa, synthetic construct
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 4 - protein, 309 aa, synthetic construct
MIFLAILDLKSLVLNAINYWGPKNNNGIQGGDFGYPISEAQIDTSIITSTHPRLIPHDLTIPQNLETIFTTTQVLTNNTDLQQSQTVSFAKKTTTTTSTSTTNGWTEGGAISDTLEEKVSVSIPFIGEGGGKNSTTIEANFAHNSSTTTFQQASTDIEWNISQPVLVPPAKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLATIEKTWYARHATLHNGKKISINNVTEMAPTSPIKTN

SEQ ID NO: 5 - DNA, 930 bp, synthetic construct
  source 1..930: mol_type = other DNA
atgatttttttggctatactagatttaaaatcactggtgttgaatgcgatcaactattggggtccgaaaaacaacaacggtattcagggtggtgattttggttacccgattagcgaaaagcagattgacaccagcattattacctctacccacccgcgtctgattccgcatgatcttacgatcccgcagaatttggaaaccatttttaccactacgcaagtcctgacgaacaacaccgacctgcagcaaagccaaaccgttagctttgctaaaaagaccactaccacgacctcgacctctacgacgaacggctggaccgagggcggcaagatctccgataccttggaggaaaaggtgtccgtgtctatcccgttcatcggcgaaggcggcggtaagaactccaccacaatcgaggcgaatttcgcgcataactcctcaacgaccacatttcaacaggcaagcaccgacatcgagtggaatatttcccaaccggttctggtgccgccgcgcaaacaggttgttgcgaccttggttattatgggtggcaatttcaccatcccgatggatttaatgaccacgatcgacagcactgagcactacagcggttacccgatcctgacctggattagcagcccggataactcctacaatggcccattcatgagctggtacttcgcgaactggccgaatctgccgagcggtttcggcccactgaacagcgacaacacggttacctataccggtagcgttgtctcgcaagtgagcgcgggggtgtatgccacggtgcgttttgatcagtatgacatccacaatctgcgcaccatcgagaagacctggtacgcccgtcatgctaccctgcacaacggtaaaaaaatcagcatcaataacgtgaccgaaatggcaccgacctctccgatcaaaaccaactaa

SEQ ID NO: 6 - DNA, 921 bp, synthetic construct
  source 1..921: mol_type = other DNA
atggctatcctggacctcaagtcgctggtgctcaacgccattaactactgggggccaaagaacaacaacggcattcaaggcggggacttcggctaccctatcagcgagaagcagattgataccagcatcattacctccacccacccgaggctcatcccacatgacctgacaattccccagaacctcgaaactatcttcaccacgacacaagttcttacaaacaatactgatctccagcaatctcagaccgtgtcatttgctaagaagactaccacgacaactgcgacctccaccacgaacggctggacggagggcgggaagatctctgacacgctggaggaaaaggtcagcgtttcgatcccgttcattggcgagggcggcggcaagaactccacaactatcgaagccaacttcgcgcacaattccagcaccacgacatcacaggaggctagcaccgatatcgagtggaacatttcgcagccagtgctcgtcccgccacggaagcaagtggtcgcaacactcgtgatcatgggcggcaatttcactattccgatggacctgatgactaccatcgattccaccgaacattacagcgggtatccaatccttacgtggattagctcaccggacaactcgtactctggccggttcatgtcttggtattttgcgaactggcctaatctgccctcagggttcggccctcttaactccgataatactgtgacctacacggggtctgttgtgtcacaggtttccgccggcgtgtacgcgacagtcagatttgaccaatatgatatccacaacctttggaccattgagaagacgtggtatgctaggcacgcaactctccataatggcaagaagattagcatcaacaatgtgacagaaatggctccaacgtcccctatcaagacaaactga

SEQ ID NO: 7 - DNA, 930 bp, synthetic construct
  source 1..930: mol_type = other DNA
atggccattctggatctgaagagcctggttctgaacgcgattaactactgggggccgaaaaataacaacggcattcagggtggcgacttcgggtacccgatctcagagaaacagatcgacacttccatcattacctttacgcacccgcgcctgattccatatgatctgacgatcccgcagaacctggaaaccatcttcacgacgactcaagttctgaccaacaacaccgatctgcagcagtcgcaaaccgtgtccttcgcaaaaaagacaacaaccacgactagcaccagcacaaccaacggctggaccgaaggggggaaaatttccgataccctcgaggagaaagtgagcgttagtatcccgtttatcggagaaggtggtggcaaaaattccaccaccattgaagccaatttcgcgcacaatagctcaaccaccaccttccaacaggccagcactgatatcgaatggaacattagccaaccggtgcttgtacctccctcgaaacaggtggtggccaccttagtcattatgggcggcaacttcacaattcctatggatctgatgaccactatcgacagtaccgaacactacagccattattcgggctatcccatcttaacttggatttcctccccggataatagctactccggtccgtttatgtcctggtatttcgctaattggcctaacctgccgagtgggtttggtccgttaaattccgacaataccgtgacctatacgggcagcgtggtatcacaagtatcggcaggcgtttatgcgaccgtccgtttcgatcaatatgacattcacaacctgcgtacgatcgagaaaacctggtacgcccgccacgccacgctgcataacggcaaaaaaattagcattaacaacgttaccgaaatggcgccgacctcgccgatcaaaaccaactga

SEQ ID NO: 8 - DNA, 930 bp, synthetic construct
  source 1..930: mol_type = other DNA
atgatttttttggctatactagatttaaaatccctggttttgaatgcgatcaactactggggtccgaagaacaacaacggcatccaaggtggtgatttcggttatccgatcagcgaagcccagattgacacctcaatcatcacctccacccatccgcgcctgattccgcacgatttgaccatcccgcaaaacttggaaaccatctttaccacgacccaggtactgaccaacaacaccgacctgcaacagagccaaaccgtgtcttttgctaaaaagactaccacgacgacctctacctctacaacgaacggctggaccgagggtggcgcaatctccgacaccctggaggaaaaggtgagcgtttctatcccgttcattggtgaaggcggcggaaaaaacagcactacgattgaagcgaacttcgcgcacaacagcagcactacgacctttcagcaagcgagcaccgatattgagtggaatatttcccagccggttctggtgccgcctgcaaagcaggtcgtggccactctggttattatgggtggtaatttcaccataccgatggatttaatgaccacgattgatagcaccgagcactacagcggctacccgatcctgacctggattagcagcccggataatagttataatggtccgttcatgagctggtacttcgccaactggccaaatcttccgagcggctttggcccactgaattctgacaacaccgtgacctataccggtagcgtggtctcgcaagtgagcgcgggtgtttatgcgaccgttcgttttgaccagtacgacatccacaatctcgcgaccatcgagaaaacctggtacgctcgtcatgctaccctgcataacggcaaaaagatcagcattaataacgttacggagatggcaccgacctcaccgattaaaaccaactaa

SEQ ID NO: 9 - DNA, 930 bp, synthetic construct
  source 1..930: mol_type = other DNA
atgattttcctcgctatcctggacctcaagtcgctggtgctcaacgccattaactactgggggccaaagaacaacaacggcattcaaggcggggacttcggctaccctatcagcgagaagcagattgataccagcatcattacctccacccacccgaggctcatcccacatgacctgacaattccccagaacctcgaaactatcttcaccacgacacaagttcttacaaacaatactgatctccagcaatctcagaccgtgtcatttgctaagaagactaccacgacaacttcaacctccaccacgaacggctggacggagggcgggaagatctctgacacgctggaggaaaaggtcagcgtttcgatcccgttcattggcgagggcggcggcaagaactccacaactatcgaagccaacttcgcgcacaattccagcaccacgacatttcagcaagctagcaccgatatcgagtggaacatttcgcagccagtgctcgtcccgccacggaagcaagtggtcgcaacactcgtgatcatgggcggcaatttcactattccgatggacctgatgactaccatcgattccaccgaacattacagcgggtatccaatccttacgtggatttcgtctcctgacaactcgtacaatggccccttcatgtcttggtattttgcgaactggcctaatctgccctcagggttcggccctcttaactccgataatactgtgacctacacggggtctgttgtgtcacaggtttccgccggcgtgtacgcgacagtcagatttgaccaatatgatatccacaaccttcgcaccattgagaagacgtggtatgctaggcacgcaactctccataatggcaagaagattagcatcaacaatgtgacagaaatggctccaacgtcccctatcaagacaaactga

SEQ ID NO: 10 - DNA, 921 bp, synthetic construct
  source 1..921: mol_type = other DNA
atggctatccttgatcttaagtcccttgtgctaaatgctatcaattattggggacctaaaaataataatggcattcaaggaggcgattttggatacccgatttcggaaaaacaaattgacacttctattattacatctactcatccgcgtctcattcctcacgatcttacaattccacaaaacttagaaacaatttttactaccactcaagttttaacgaataatacagatttgcaacaatcacaaacggtttcgtttgcgaagaagacaacaactaccacagcgacttccaccacaaacggttggactgaagggggaaaaatttctgacactttagaagagaaggtttcagtctcgatcccttttataggagaagggggtggaaagaactcaacgacaatagaagcaaacttcgcacacaactcttcgactactacatcccaggaagctagcacagatatagaatggaacatctcacagcctgtgcttgtgccgccaagaaaacaagttgttgccactttggtcataatgggaggtaacttcactatcccaatggatctaatgacaacaattgattctaccgaacactattccggttacccgatattaacctggatatcaagtccagataattcatactctggaagattcatgtcatggtatttcgctaactggcctaatcttccttctggattcggacctttaaactctgataatacggtgacatatactggaagcgtggtgtctcaagtgagcgctggtgtgtatgcaacggtcagattcgaccagtacgatatacacaatttatggacgattgaaaagacatggtatgctagacatgctactctccacaacggaaagaaaatcagcattaataacgttaccgaaatggctccaactagtcctatcaaaactaactga

SEQ ID NO: 11 - DNA, 930 bp, synthetic construct
  source 1..930: mol_type = other DNA
atggcaattctagatttaaagagcttagtcttaaatgccataaactactggggaccgaaaaataacaatgggattcaaggaggtgatttcggatacccaatttctgagaagcaaatcgatacatctattataacatttactcatccaagacttatcccgtacgatttaaccatccctcaaaacctcgaaactatctttactacgacacaggttttgacaaacaacaccgatcttcagcagagccagacagttagtttcgccaagaagactactactaccacctctacttcaaccactaacggatggaccgagggtgggaagatttccgatactcttgaggaaaaagtgagcgttagcataccgtttatcggagaaggtggaggaaagaattctacgactatcgaggctaatttcgctcacaactcttcgactacgacttttcaacaagcatctactgatattgagtggaatatttctcagcctgtattggttccaccttcaaaacaagttgtggctacattagtcatcatggggggtaactttacgattcctatggacctgatgacaacaattgattcaactgaacattactcgcattattcaggttacccaatcctcacatggatttcaagcccagacaacagctattcgggaccatttatgtcatggtacttcgcaaattggcccaatctgccgagtggatttggtcctctcaattcggataacacagttacatacactggttcggttgttagtcaagtaagtgctggagtgtatgcaaccgtcagattcgatcagtatgatattcataatcttaggactatcgagaaaacatggtatgctagacacgcgacgcttcataatggaaaaaagatttccattaacaatgtgactgagatggcaccaacgtctccgattaaaactaattga

SEQ ID NO: 12 - DNA, 930 bp, synthetic construct
  source 1..930: mol_type = other DNA
atgatcttcctcgcgattttggatctaaagtcacttgtactcaatgcgattaattattggggacctaaaaacaataatggcattcaaggaggagatttcggttacccaatatctgaagctcagatcgatacatccattattaccagcactcatcctaggctcattcctcatgaccttactattccacagaaccttgaaactattttcacaacgacgcaagtgttgacaaataacaccgacttgcaacaatctcagactgtttcttttgctaagaaaaccactacaacaacatctacttcaaccacgaatggatggacggaaggaggtgctatatccgacaccttggaagaaaaggtgtcggtgtcaatccctttcattggagaaggcggtgggaagaactctactacaattgaagctaactttgctcataattcttcgactacaacttttcagcaagcttccacggatatcgaatggaacatttcacagcctgttttggtgccacccgcaaaacaggttgtggcaactttggtgattatgggtgggaattttactatccctatggatcttatgacgactattgattctactgagcactacagcggatatcctattcttacctggatctcttcacctgacaatagctataacgggccttttatgagttggtattttgcaaattggcccaacctcccaagtggattcgggccacttaactctgataatacagttacctacactggttccgtggttagtcaagtgtcggctggtgtgtatgctaccgtgcgttttgatcaatatgatattcataacctcgctacaattgaaaagacttggtacgctcgacacgcaacgctccacaatggtaaaaaaatctcgattaacaatgttaccgagatggcacccactagtccaatcaagactaactga

SEQ ID NO: 13 - protein, 653 aa, synthetic construct
MANPNNRSEHDTIKVTPNSELQTNHNQYPLADNPNSTLEELNYKEFLRMTEDSSTEVLDNSTVKDAVGTGISVVGQILGVVGVPFAGALTSFYQSFLNTIWPSDADPWKAFMAQVEVLIDKKIEEYAKSKALAELQGLQNNFEDYVNALNSWKKTPLSLRSKRSQDRIRELFSQAESHFRNSMPSFAVSKFEVLFLPTYAQAANTHLLLLKDAQVFGEEWGYSSEDVAEFYRRQLKLTQQYTDHCVNWYNVGLNGLRGSTYDAWVKFNRFRREMTLTVLDLIVLFPFYDIRLYSKGVKTELTRDIFTDPIFLLTTLQKYGPTFLSIENSIRKPHLFDYLQGIEFHTRLRPGYFGKDSFNYWSGNYVETRPSIGSSKTITSPFYGDKSTEPVQKLSFDGQKVYRTIANTDVAAWPNGKVYLGVTKVDFSQYDDQKNETSTQTYDSKRNNGHVSAQDSIDQLPPETTDEPLEKAYSHQLNYAECFLMQDRRGTIPFFTWTHRSVDFFNTIDAEKITQLPVVKAYALSSGASIIEGPGFTGGNLLFLKESSNSIAKFKVTLNSAALLQRYRVRIRYASTTNLRLFVQNSNNDFLVIYINKTMNKDDDLTYQTFDLATTNSNMGFSGDKNELIIGAESFVSNEKIYIDKIEFIPVQL

SEQ ID NO: 14 - DNA, 1962 bp, synthetic construct
  source 1..1962: mol_type = other DNA
atggcgaaccccaacaataggtccgagcacgacacgatcaaggtcacacccaactccgagctgcagactaatcacaatcagtacccactcgccgacaaccccaatagcacgctcgaggagctgaactacaaggagttcctccgcatgacggaggactcctccacagaggtgctggataattctactgtcaaggacgcggttggcaccgggatctcagtggtcgggcagattctcggcgttgtgggggtcccattcgctggcgctctgacgtccttctaccagagcttcctcaacacaatctggccatccgatgccgacccttggaaggccttcatggcgcaggtggaggtcctgatcgacaagaagattgaggagtacgccaagtcgaaggctctcgctgagctgcagggcctccagaacaatttcgaggactacgtgaacgcgctcaattcatggaagaagaccccactctccctgaggagcaagcgctcgcaggaccgcatcagggagctgttctcgcaggcggagtctcacttcaggaactctatgccctcattcgctgtgagcaagttcgaggtcctcttcctgccaacgtacgcccaggccgcgaacacacatctcctgctcctgaaggacgctcaggtgttcggggaggagtggggctactcgtctgaggatgtcgccgagttctaccgcaggcagctcaagctgacgcagcagtacacagaccactgcgttaactggtacaatgtgggcctgaacgggctcaggggctctacgtacgatgcgtgggtgaagttcaatcggttccggcgcgagatgaccctgacggtcctcgatctgatcgttctcttccccttctacgacattcgcctgtactcgaagggcgtcaagacggagctcacaagggatatcttcactgacccaattttcctcctgaccacgctccagaagtacggccctaccttcctgtcaatcgagaactccattcgcaagccgcacctgttcgactacctccagggcatcgagttccatacccggctgcgccccgggtacttcggcaaggattctttcaactactggtcagggaattacgtggagacacgcccgtcaatcggctcatccaagacaattacttcaccgttctacggggataagtccacagagcccgtccagaagctctccttcgacggccagaaggtttaccgcactatcgccaacaccgatgtggctgcctggccgaatgggaaggtctacctgggcgttacaaaggtggacttcagccagtacgacgatcagaagaacgagacatccacgcagacatacgattctaagaggaacaatggccatgtgagcgcgcaggattcgatcgaccagctcccgcccgagacaactgacgagcctctggagaaggcttacagccaccagctgaactacgccgagtgcttcctcatgcaggacaggcggggcaccatcccgttcttcacttggacccatcggtcggtcgatttcttcaacactatcgacgctgagaagattacccagctccctgtcgttaaggcgtacgctctgagctcgggcgcttccatcattgagggcccagggttcactggcgggaacctcctgttcctcaaggagtcttcaaatagcatcgctaagttcaaggttacgctgaactcggctgctctcctgcagaggtaccgggtccgcattcgctacgcgtccaccacgaacctcaggctgttcgttcagaatagcaacaatgacttcctcgtgatctacattaacaagaccatgaataaggacgatgacctgacttaccagaccttcgacctcgcgacaactaactccaatatggggttcagcggcgataagaacgagctgatcattggcgctgagtccttcgtgtccaacgagaagatctacattgataagattgagttcattcccgtccagctctga

SEQ ID NO: 15 - DNA, 1992 bp, Zea mays
  source 1..1992: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 16 - DNA, 253 bp, Agrobacterium tumefaciens
  source 1..253: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 17 - DNA, 1026 bp, Salmonella enterica
  source 1..1026: mol_type = genomic DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 18 - DNA, 20 bp, synthetic construct
  source 1..20: mol_type = other DNA
cagggtgtcacgttgcaaga

SEQ ID NO: 19 - DNA, 20 bp, synthetic construct
  source 1..20: mol_type = other DNA
ccgctcgtctggctaagatc

SEQ ID NO: 20 - DNA, 24 bp, synthetic construct
  source 1..24: mol_type = other DNA
tgcctgaaaccgaactgcccgctg